SEQUENCE LISTING (7 sequences)

SEQ ID NO: 1 - protein, 811 aa, Homo sapiens
MEAARALRLLLVVCGCLALPPLAEPVCPERCDCQHPQHLLCTNRGLRVVPKTSSLPSPHDVLTYSLGGNFITNITAFDFHRLGQLRRLDLQYNQIRSLHPKTFEKLSRLEELYLGNNLLQALAPGTLAPLRKLRILYANGNEISRLSRGSFEGLESLVKLRLDGNALGALPDAVFAPLGNLLYLHLESNRIRFLGKNAFAQLGKLRFLNLSANELQPSLRHAATFAPLRSLSSLILSANSLQHLGPRIFQHLPRLGLLSLRGNQLTHLAPEAFWGLEALRELRLEGNRLSQLPTALLEPLHSLEALDLSGNELSALHPATFGHLGRLRELSLRNNALSALSGDIFAASPALYRLDLDGNGWTCDCRLRGLKRWMGDWHSQGRLLTVFVQCRHPPALRGKYLDYLDDQQLQNGSCADPSPSASLTADRRRQPLPTAAGEEMTPPAGLAEELPPQPQLQQQGRFLAGVAWDGAARELVGNRSALRLSRRGPGLQQPSPSVAAAAGPAPQSLDLHKKPQRGRPTRADPALAEPTPTASPGSAPSPAGDPWQRATKHRLGTEHQERAAQSDGGAGLPPLVSDPCDFNKFILCNLTVEAVGADSASVRWAVREHRSPRPLGGARFRLLFDRFGQQPKFHRFVYLPESSDSATLRELRGDTPYLVCVEGVLGGRVCPVAPRDHCAGLVTLPEAGSRGGVDYQLLTLALLTVNALLVLLALAAWASRWLRRKLRARRKGGAPVHVRHMYSTRRPLRSMGTGVSADFSGFQSHRPRTTVCALSEADLIEFPCDRFMDSAGGGAGGSLRREDRLLQRFAD

SEQ ID NO: 2 - protein, 809 aa, mouse
MEGVGAVRFWLVVCGCLAFPPRAESVCPERCDCQHPQHLLCTNRGLRAVPKTSSLPSPQDVLTYSLGGNFITNITAFDFHRLGQLRRLDLQYNQIRSLHPKTFEKLSRLEELYLGNNLLQALVPGTLAPLRKLRILYANGNEIGRLSRGSFEGLESLVKLRLDGNVLGALPDAVFAPLGNLLYLHLESNRIRFLGKNAFSQLGKLRFLNLSANELQPSLRHAATFVPLRSLSTLILSANSLQHLGPRVFQHLPRLGLLSLSGNQLTHLAPEAFWGLEALRELRLEGNRLNQLPLTLLEPLHSLEALDLSGNELSALHPATFGHQGRLRELSLRDNALSALSGDIFAASPALYRLDLDGNGWTCDCRLRGLKRWMGNWHSQGRLLTVFVQCRHPPALRGKYLDYLDDQLLQNGSCVDPSPSPTAGSRQWPLPTSSEEGMTPPAGLSQELPLQPQPQPQQRGRLLPGVAWGGAAKELVGNRSALRLSRRGPGPHQGPSAAAPGSAPQSLDLHEKPGRGRHTRANLSQTEPTPTSEPASGTPSARDSWQRAAKQRLASEQQESAVQSVSGVGLPPLVSDPCDFNKFILCNLTVEAVSANSASVRWAVREHRSPRPQGGARFRLLFDRFGQQPKFQRFVYLPERSDSATLHELRGDTPYLVCVEGVLGGRVCPVAPRDHCAGLVTLPEAGGRGGVDYQLLTLVLLAVNALLVLLALAAWGSRWLRRKLRARRKGGAPVHVRHMYSTRRPLRSMGTGVSADFSGFQSHRPRTTVCALSEADLIEFPCDRFMDSTGGGTSGSLRREDHLLQRFAD

SEQ ID NO: 3 - DNA, 2436 bp, Home sapiens
atggaggctgcccgcgccttgcgcctcctgctcgtggtgtgcggctgcctcgcgctcccgccgctggccgagcccgtgtgcccggagcgctgcgactgccagcatccccagcatctcctgtgcaccaacagggggctccgcgtagtgcccaagaccagctcgctgccgagcccccacgacgtgctcacctacagcctcggcggcaacttcataaccaacatcacggccttcgacttccaccgtctggggcagctcagacggctggacctgcagtacaaccagatccgctctctgcaccccaagaccttcgagaagctctcgcggctggaagagctgtacctggggaacaacctcttgcaggcgctcgccccgggcacgctggccccgctgcgcaagctgcgcatcctctacgccaacgggaacgagatcagccgcctaagccgcggctccttcgagggcctggagagtctagtcaagctgcggctggacgggaacgccctgggggcgctgccggacgcggtcttcgctcccttgggcaacctgctctacctacatctggagtccaaccggatccgctttctgggcaagaacgccttcgcccagctaggcaagctgcgcttcctcaacctctctgccaacgagctacagccctccctgcgccacgcggccaccttcgcaccgctgcgctccctctcctccctcatcctctcggccaacagcctgcagcacctcgggccgcgcatcttccagcacctgccacgtctcggcctgctctcgctcaggggcaaccagctcacgcacctcgcgcctgaggccttttggggcttggaggccctgcgcgagctgcgcctggagggtaatcggctgagccagctgccaactgcgctgctggagcctctgcacagcctggaggcgctggacctgagcggcaatgagctgtccgccctgcacccggccaccttcggccacctgggccggctgcgcgagctcagcctgcgcaacaacgcgctcagcgccctatccggggacatcttcgccgccagcccagccctttatcggctggatctagacggcaacggctggacctgcgactgccggctgcgaggcctgaagcgctggatgggcgactggcactcgcagggccggctcctcactgtcttcgtgcagtgtcgccaccccccggccctgcgaggcaaatacctggattacctggatgaccagcagctgcaaaatggatcctgcgcggatccctcgccctcagcttccctgaccgctgaccgcaggcggcagcccctacccacggccgcaggggaggagatgacgccacctgcaggtctcgcggaggagctgccgccgcagccgcagctccagcagcaggggcgatttctagctggggtggcctgggatggggccgccagggagctggtaggcaaccgcagcgctctaaggctgagtcggcggggcccgggcctccagcagcccagcccctccgtcgctgccgccgcgggcccggctccacagtccctagacctgcacaagaagccccagcggggccgtccgactcgggcagatcccgccctcgcggagcccaccccaacggcctctcctggctctgcgccatcgcccgccggcgacccctggcagcgcgcgacgaagcatcgtctgggcacggagcaccaggagcgtgccgcccagtccgacggtggggccgggctgccgccgctggtgtccgacccatgcgacttcaacaagttcattctgtgcaacctgacggtggaggcggtgggcgcagacagcgcctcggtgcgctgggccgtgcgcgagcaccgcagtccccggccgctgggcggcgcgcgcttccgcctgctctttgaccgctttggccagcagcccaagttccaccgcttcgtctacctgcctgagagcagcgactcggccacgctgcgcgagctgcgcggggacaccccctacctggtgtgcgtggagggcgtgcttgggggccgtgtctgccctgtggctccccgggaccactgcgcggggctggtcaccctaccggaggccgggagccggggcggcgtcgactaccagctgctgaccttggccctgctgacggtcaacgcgctgctggtgctcctggccttggcggcctgggcgtctcgctggctgcgtaggaaactgcgggctaggcggaagggcggggccccggtccacgttcggcacatgtactccacccgacggcccctgcgctccatgggcaccggcgtgtccgccgacttctcgggattccagtcgcaccggccacgcaccaccgtgtgcgcgctcagtgaggcggacctcatcgaattcccctgcgaccgcttcatggacagtgcgggcggcggcgcgggcggcagcctgagacgggaggaccgtctcctgcagcgatttgccgactag

SEQ ID NO: 4 - DNA, 2430 bp, mouse
atggagggtgtgggtgccgtgcgcttctggctcgtggtgtgcggctgcctggcgttcccgccgcgggccgagtccgtgtgcccggagcgctgcgactgccagcacccccaacatctcctatgcaccaacaggggacttcgcgctgtacccaagaccagctcgctgcctagtccccaggacgtgctcacctacagcctaggcggcaacttcatcaccaacatcaccgccttcgacttccaccgtctggggcagctcagacggctggatctgcagtacaaccagatccgttctctccaccccaagaccttcgagaagctctcacgactagaggagctgtacctggggaacaacctcttgcaggcgctcgttcctggcacgttggctccgctgcgcaagttgcgcatcctttacgccaacggcaacgagattggacgcctgagtcgcgggtccttcgagggcctggagagtttggtcaagctacggctggacgggaacgtgctgggagcgctgccggacgcggtcttcgctcccctgggcaacctgctctacctacacctggaatctaaccggatccgttttttgggcaagaacgccttcagccagctgggtaagcttcgattcctcaatctctcggccaacgagctgcaaccctctctgcgccacgcggccacctttgtccctctgcgctccctctctactctcatcctctcagccaacagcctacagcacctcggcccccgcgtcttccagcacctgccacgcctaggcctgctctccctcagtggaaaccagctcacgcacctcgccccagaggccttttggggactggaggccctacgtgagctgcgcctggaaggtaaccggctgaaccagctccccctgacactgctggagcctctgcacagcttggaggcactagacctgagtggcaatgagctttctgctctgcaccctgccacctttggccaccagggccggctccgtgagctcagcctgcgcgacaacgcgctcagcgccctttctggggacatctttgccgccagtccggctctctaccggctggatctagacggcaatggttggacctgcgactgccggttgcggggtctgaaacgctggatgggcaactggcattctcagggtcgcctccttacggtcttcgtgcagtgtcgccaccccccggccctgcggggcaagtacctggattacctggatgaccaactgctgcagaatgggtcttgcgtggatccctcaccttcccctacagcaggcagtaggcagtggccgctacccacatcgtcagaagaggggatgacgccccctgcaggcctctcgcaggagctaccactgcagccgcagccgcagccccagcagcggggaagacttctaccaggagtggcctggggtggggctgccaaggaactggtgggcaaccgcagcgcattaaggttgagccggcggggaccaggtccccatcagggcccctctgctgcggctccgggctctgccccacagtccttggacctgcacgagaagccggggagaggacgtcacacccgagccaatctctcccagacggagcccaccccgacgtctgagcccgcctctgggacaccatctgccagagacagctggcagcgcgcagcaaagcagcgcctagcgtccgagcagcaagagagcgcagtccagtccgttagtggggttggtttgccacctctggtgtctgacccctgtgacttcaacaaattcattctgtgcaacctgacagtagaggcggtgagcgccaacagcgcctcggtgcgctgggcggttcgcgaacaccgcagtccccggccgcagggcggcgcacgcttccgcctgcttttcgaccgctttggccagcagcccaagttccagcgcttcgtctacctgccggagcgcagcgactcggccacgctgcacgagctgcgcggagacaccccctacctagtatgcgtggagggtgtgctcgggggtcgcgtttgccccgtggccccccgagaccactgcgcagggttagtaaccctgccagaggcaggaggtcgaggtggcgtcgactaccagctcctgaccttggtcttgctggctgtcaacgcgcttctggtgctcctggcactggcagcctggggatcgcgatggctgcggaggaagctgcgtgccaggcggaagggaggggccccggtccacgttcgccacatgtactccacccgacggccactgcgctccatgggcactggtgtgtccgcggacttctcaggcttccagtcgcaccgtccccgcaccaccgtgtgcgcgctcagcgaggccgacctcattgagttcccctgcgaccgcttcatggacagcaccggcggtggtaccagtggcagcttgaggcgggaggatcacctcttgcaaagattcgctgactag

SEQ ID NO: 5 - DNA, 29 bp, artificial sequence
  primer consisting of short complementary sequences from the TLR14 nucleotide sequence
gcaagcttatggaggctgcccgcgccttg

SEQ ID NO: 6 - DNA, 54 bp, artificial sequence
  primer consisting of short complementary sequences from the TLR14 nucleotide sequence
gcgatatcggcctaagcgtagtctgggacgtcgtatgggtagtcggcaaatcgc

SEQ ID NO: 7 - protein, 16 aa, artificial sequence
  amino acids from the C-terminus of TLR14
CGSLRREDRLLQRFAD

(19) United States
(12) Patent Application Publication
(10) Pub. No.: US 2011/0200605 A1
(43) Pub. Date: Aug. 18, 2011

(54) Polypeptides and use thereof

(75) Inventors: Luke Anthony John O'Neill, Co. Dublin (IE); Aisling Dunne, Dublin 6 (IE)

(73) Assignees: The Provost, Fellows and Scholars of the College of the Holy and Undivided Trinity of Queen; Elizabeth, Near Dublin

(21) Appl. No.: 12/952,869

(22) Filed: Nov. 23, 2010

Related U.S. Application Data
(63) Continuation of application No. 11/918,642, filed on Oct. 17, 2007, now abandoned.
(60) Provisional application No. 60/672,051, filed on Apr. 18, 2005.

(51) Int. Cl. A61K 39/395; A61K 38/16; A61K 39/00; C07K 14/435; C12Q 1/68; C07H 21/04; C12N 15/63; C12N 5/10; C12P 21/06; C07K 16/18; A61P 29/00; A61P 37/00

(52) U.S. Cl. 424/139.1; 514/21.2; 424/185.1; 530/350; 435/6.13; 536/23.5; 435/320.1; 435/325; 435/69.1; 530/387.9

(57) ABSTRACT

An isolated polypeptide comprises an amino acid sequence of SEQ ID No. 1 or 2 or a variant or fragment thereof. The variant may comprise an amino acid sequence that is at least 70% or 95% identical to the amino acid sequence of SEQ ID No. 1 or 2. A fragment thereof may be a peptide comprising at least 12 contiguous amino acids of SEQ ID No. 1 or 2. The polypeptide exhibits toll-like receptor activity. The TLR has been named TLR1 4. TLR receptors recognise a range of ligands and activate a series of signalling pathways that lead to the induction of immune and inflammatory genes.

### INTRODUCTION

The Toll-like receptor/Interleukin-1 receptor (TLR) superfamily plays a central role in inflammation and the host response to bacterial infection. Members of the TLR family are characterised by a cytosolic domain termed the Toll-IL-1R (TIR) domain and an extracellular region consisting of a series of leucine rich repeats. Occupation of toll-like receptors by various microbial components leads to the expression of a large number of proinflammatory proteins such as inducible cyclooxygenase, adhesion molecules and chemokines. Ten human TLRs have been identified to date. TLR4, the first TLR to be discovered, is essential for the response to bacterial lipopolysaccharide (LPS) (1,2). TLR2 couples with TLRs 1 and 6 to recognise diacyl- and triacyl-lipopeptides respectively. TLR5 recognises and responds to bacterial flagellin (3) and TLR9 is required for recognition of unmethylated CpG motifs which are present in bacterial DNA (4). TLRs 11, 12 and 13 have recently been described in mice but they have no human orthologs (5, 6). Stimulation of TLRs with the appropriate ligands leads to activation of the transcription factor NF-κB and also the mitogen-activated protein kinases (MAPKs), p38, c-jun N terminal kinase (JNK) and p42/p44.

The activation of NF-κB is dependent on MyD88, a cytoplasmic TIR domain-containing adapter protein (7, 8, 9). MyD88 acts as an adapter protein for the entire TLR family with the exception of TLR3 which recruits the adapter protein TRIP (10). In addition to activating NF-κB, TRIF is also required for the induction of genes dependent on the transcription factor Interferon Regulatory Factor 3 (IRF3) (11). This pathway is referred to as the MyD88-independent pathway and has been shown to be important for evading pathogens of viral origin (12). Another TIR adapter protein, MyD88 Adapter-like (Mal, also known as TIRAP) is involved in the MyD88 dependent pathway (13, 14) and is required specifically for TLR2 and TLR4 mediated signalling (15, 16).

During infection, occupation of TLRs by various ligands leads to the production of inflammatory mediators such as cytokines and chemokines and the activation of immune effector cells. This co-ordinated response is designed to clear invading pathogens, however, in many instances bacterial products activate an uncontrolled network of host derived mediators which can lead to multi-organ failure, cardiovascular collapse and eventually death. This condition, referred to as sepsis, is the major cause of deaths in intensive care units of hospitals and continues to increase worldwide. Antagonists for TLR proteins might therefore be useful tools to counteract the harmful effects of over-active immune responses. Interruption of TLR4 signaling is being closely examined as a means of counteracting the toxic effects of LPS. Current therapies include neutralizing antibodies to TLR4 and its co-receptor CD14 and also synthetic lipid A analogues which compete with LPS for binding to the receptor (17, 18).

As well as sepsis, therapies are also being aimed at other TLRs as a means of combating viral infections. For example, the TLR7 agonist, imiquimod, has been used successfully in the treatment of genital herpes caused by the human papilloma virus (19). In the case of autoimmune diseases, TLR agonists have been considered as a means of shifting adaptive Tₕ2 responses to Tₕ1 immune responses which would subsequently prevent the development of allergy. A more long-term goal will involve the development of therapeutics aimed at downstream components of the TLR signalling pathway. It is therefore crucial that all aspects of TLR signalling are fully understood.

The identification of further members of the TLR family or aspects of the TLR signalling pathway have valuable pharmaceutical potential.

### STATEMENT OF INVENTION

According to the invention there is provided an isolated polypeptide comprising an amino acid sequence of SEQ ID No. 1 or a variant or fragment thereof.

The invention also provides an isolated polypeptide comprising amino acid sequence SEQ ID No. 2 or a variant or fragment thereof.

In one embodiment of the invention the variant comprises an amino acid sequence that is at least 70% identical to the amino acid sequence of SEQ ID No. 1 or 2. In another embodiment of the invention the variant Comprises an amino acid sequence that is at least 75%, at least 80%, at least 85%, at least 90%, at least 95%, at least 96%, at least 97%, at least 98%, at least 99%, at least 99.5% identical to the amino acid sequence of SEQ ID No. 1 or 2.

In one embodiment of the invention the variant comprises a deletion or insertion modification. The variant may also comprise a post translation modification.

In one embodiment of the invention the fragment is a peptide comprising at least 12 contiguous amino acids of SEQ ID No. 1 or 2.

In one embodiment of the invention the polypeptide as hereinbefore described exhibits Toll-like receptor activity. The Toll-like receptor activity may be TLR14 activity.

In one embodiment of the invention the polypeptide exhibits immunomodulatory activity.

The invention also provides a polynucleotide encoding a polypeptide as hereinbefore described.

The invention further provides an isolated polynucleotide comprising a nucleic acid sequence SEQ ID No. 3 or variant or fragment thereof or a sequence complementary thereto.

The invention also provides an isolated polynucleotide comprising a nucleic acid sequence SEQ ID No. 4 or variant or fragment thereof or a sequence complementary thereto.

In one embodiment of the invention the polynucleotide comprises a nucleic acid sequence that is at least 70% identical to the nucleic acid sequence of SEQ ID NO. 3 or 4.

In another embodiment of the invention the fragment comprises at least 17 contiguous nucleic acids of SEQ ID No. 3 or 4.

In one embodiment of the invention the polynucleotide exhibits at least 80% identity top natural cDNA encoding said segment.

In one embodiment of the invention the polynucleotide encodes a Toll-like receptor or peptide or fusion protein thereof.

The invention also provides a recombinant nucleic acid comprising a nucleic acid sequence of SEQ ID No. 3 SEQ ID No. 4 or variant or fragment thereof or a sequence complementary thereto.

The invention further provides a purified protein or peptide comprising an amino acid sequence of SEQ ID No. 1 or 2 or a variant or fragment thereof. Preferably a fragment of the protein or peptide comprises at least 12 contiguous amino acids of SEQ ID No. 1 or 2.

In one embodiment of the invention the protein or peptide is of mammalian origin. The protein may be of human origin.

In one embodiment of the invention the protein or peptide has a molecular weight of at least 100 kDa. The protein or peptide may be in glycosylated form.

One embodiment of the invention provides a recombinant protein or peptide comprising an amino acid sequence of SEQ ID No. 1 or 2.

The protein or peptide of the invention may exhibit Toll-like receptor functionality/activity.

The invention also provides a protein comprising an amino acid sequence selected from SEQ ID No. 1 or 2 or a variant or fragment thereof. The protein may be a Toll-like receptor protein,especially TLR14.

The invention also provides an antigenic fragment of a protein or peptide of the invention.

The invention also provides a recombinant vector comprising a polynucleotide as hereinbefore described. The invention also provides a host cell comprising the recombinant vector. The invention further provides a gene therapy agent comprising the recombinant vector as an active ingredient.

One aspect of the invention provides an adjuvant comprising a polypeptide as hereinbefore described.

The invention also provides a fusion compound or chimeric molecule comprising any one or more of
a protein comprising an amino acid sequence of SEQ ID No. 1 or 2 or a fragment or variant thereof; and
a detection or purification tag.

In one embodiment of the invention the detection or purification tag is selected from any one ore more of a FLAG sequence, His6 sequence, Ig sequence and a heterologous polypeptide of another receptor protein.

The invention also provides a ligand/receptor complex comprising a recombinant or synthetically produced protein comprising an amino acid sequence of SEQ ID No. 1 or 2 and a TLR ligand. Preferably the TLR ligand is a CpG nucleic acid.

The invention also provides an immunogen comprising an antigenic determinant of a protein as hereinbefore described.

The invention further provides a monoclonal or polyclonal antibody or fragment thereof that specifically binds to an epitope of a polypeptide or a protein or peptide as hereinbefore described. The antibody may be prepared in an immobilised form. The antibody may be immobilised by conjugation or attachment to a bead, a magnetic bead, a slide, or a container. The antibody may be immobilised to cyanogen bromide-activated sepharose or absorbed to polyolefin surfaces with or without glutaraldehyde cross-linking.

The invention also provides a method for identifying compounds which modulate Toll-like receptor activity comprising the steps of:
contacting a polypeptide comprising an amino acid sequence of SEQ ID No. 1 or 2 or variant or fragment thereof with a test sample;
monitoring for markers of Toll-like receptor activity; and
identifying the compounds which modulate Toll-like receptor activity.

In one embodiment of the invention the markers of Toll-like receptor activity comprise any one or more of:
(i) NFkappaB activation
(ii) NFkappaB protein or polynucleotide encoding the same
(iii) IRF3 protein or polynucleotide encoding the same
(iv) p38 protein or polynucleotide encoding the same
(v) IKKs protein or polynucleotide encoding the same
(vi) RANTES protein or polynucleotide encoding the same
(vii) TLR4 protein or polynucleotide encoding the same or
(viii) any pro-inflammatory or inhibitory cytokine.

In one embodiment the method comprises the step of determining the difference in the amount relative to the test sample of at least 2 of each of (i) to (viii).

In another embodiment the method comprises the step of determining the difference in the amount relative to the test sample of at least 3 of each of (i) to (viii).

In one case the amount relative to the test sample of protein is determined. Alternatively the amount relative to the test sample of mRNA is determined using nucleic acid microarrays. The Toll-like receptor activity may be TLR14 activity.

In one embodiment of the invention a compound which activates or inhibits TLR activity is identified by determining the amount, expression, activity or phosphorylation relative to the test sample of a least one or more of:
(i) NFkappaB activation
(ii) NFkappaB protein or polynucleotide encoding the same
(iii) IRF3 protein or polynucleotide encoding the same
(iv) p38 protein or polynucleotide encoding the same
(v) IKKs protein or polynucleotide encoding the same
(vi) RANTES protein or polynucleotide encoding the same
(vii) TLR4 protein or polynucleotide encoding the same or
(viii) any pro-inflammatory or inhibitory cytokine.

In another embodiment a compound capable of modulating TLR activity is identified by a method as hereinbefore described.

The invention also provides a pharmaceutical composition comprising a compound of the invention and a pharmaceutically acceptable carrier.

The invention also provides a pharmaceutical composition comprising:
a reagent or compound that modulates the activity of a TLR14 polypeptide comprising an amino acid sequence of SEQ ID No. 1 or 2 or a polynucleotide comprising a nucleic acid of SEQ ID No. 3 or 4; and
a pharmaceutically acceptable carrier.

In one embodiment on the invention the reagent is a TLR14 agonist or antagonist.

Preferably the carrier compound is an aqueous compound selected from any one or more of water, saline and buffer. The composition may be in a form for oral, rectal, nasal, topical or parenteral administration.

In one embodiment of the invention the compound or composition as is used in the preparation of a medicament for the treatment of any one or more of allergic disease, autoimmune disease, inflammatory disease, cardiovascular disease, CNS disease, neoplastic disease and infectious disease, and/or immune-mediated disorder.

In one embodiment of the invention the disorder is selected from any one or more of sepsis or acute inflammation induced by infection, trauma or injury, chronic inflammatory disease, graft rejection or graft versus host disease, Crohn's disease, inflammatory bowel disease, multiple sclerosis, type 1 diabetes or rheumatoid arthritis, asthma or atopic disease and allergic encephalomylitis.

Other immune-mediated disorders include any one or more of diabetes mellitus, arthritis (including rheumatoid arthritis, juvenile rheumatoid arthritis, osteoarthritis, psoriatic arthritis), atherosclerosis, myasthenia gravis, systemic lupus erythematosis, autoimmune thyroiditis, dermatitis (including atopic dermatitis and eczematous dermatitis), Sjogren's Syndrome, including keratoconjunctivitis sicca secondary to Sjogren's Syndrome, alopecia areata, allergic responses due to arthropod bite reactions, aphthous ulcer, iritis, conjunctivitis, keratoconjunctivitis, ulcerative colitis, asthma, allergic asthma, cutaneous lupus erythematosus, scieroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, primary biliary cirrhosis, uveitis posterior, interstitial lung fibrosis, Alzheimers disease or coeliac disease.

The invention further provides an agonist or antagonist compound to a TLR14 polypeptide having an amino acid sequence of SEQ ID No. 1 or 2 or a variant

The invention also provides a method of modulating the physiology or development of a cell or tissue culture cells comprising contacting the cell with an agonist or antagonist of a mammalian TLR14.

The invention further provides a method of screening compounds capable of inhibiting or promoting NF-κB activation comprising the steps of:
providing a cell with a gene encoding a protein as hereinbefore described and a component that provides a detectable signal associated with activation of NF-κB;
culturing a transformed cell under conditions providing the expression of the gene in the transformed cell;
contacting the transformed cell with one or more compounds for screening;
measuring the detectable signal; and
isolating or identifying the activator compound or inhibitor compound by measuring the detectable signal.

In one embodiment the method includes the step of
optimising the isolated or identified compound as a pharmaceutical compound.

The invention also provides a kit for screening a compound capable of modulating Toll like receptor activity comprising:
a cell comprising a gene encoding a protein of the invention and a component that provides a detectable signal upon activation of NFκB; and
reagents for measuring the detectable signal.

In one embodiment of the invention the gene encodes a Toll-like receptor TLR14.

The invention also provides use of a polypeptide comprising a fragment or variant of the amino acid sequence of SEQ ID No. 1 or 2 which is capable of inhibiting the activity of TLR14 having the amino acid sequence of SEQ ID No. 1 or 2 in the manufacture of a medicament for the treatment of an immune or inflammatory disorder.

The invention also provides use of a polypeptide, polynucleotide or compound as hereinbefore described, in the manufacture of an adjuvant or vaccine formulation.

The present invention is directed to a novel mammalian receptor, Toll-like receptor 14 (TLR14) and its biological activities. It includes nucleic acids coding for the polypeptide and methods for its production and use. The nucleic acids of the invention are characterized in part by their homology to cloned complimentary DNA (cDNA) sequences enclosed herein.

In certain embodiments, the invention comprises a composition of matter selected from the group of: a substantially pure or recombinant TLR14 protein or peptide exhibiting identity over at least 12 amino acids to SEQ ID No. 1 or 2, a natural sequence of TLR14 of SEQ ID No. 1 or 2, a fusion protein comprising TLR14 sequence composition of matter: novel TLR (TLR14). In specific embodiments the composition of matter is TLR14 which comprises a mature sequence of SEQ ID No. 1 or 2, or lacks a post-translational modification, or the composition of matter may be a protein or peptide which is from a warm blooded animal selected from a mammal including a primate, such as a human, comprising at least one polypeptide of SEQ ID No. 1 or 2; is glycosylated, has a molecular weight of at least 100 kDa with natural glycosylation, is a synthetic polypeptide; is conjugated to another chemical moiety; is a 5-fold of less substitution from natural sequence or is a deletion or insertion variant from a natural sequence. In specific embodiments, the TLR, antigenic fragment of TLR, antibody to TLR, antibody fragment to TLR, antibody to a TLR ligand also includes an immobilised form. Immobilisation may be by conjugation or attachment to a bead, a magnetic bead, to a slide, or to a container. Immobilisation may be to cyanogen bromide-activated sepharose by methods well-known in the art, or absorbed to polyolefin surfaces with or without glutaraldehyde cross-linking.

Other embodiments include a composition comprising a sterile TLR14 protein or peptide, or the TLR14 protein or peptide and a carrier wherein the carrier is an aqueous compound including water, saline, and/or buffer, and/or formulated for oral, rectal, nasal, topical or parenteral administration.

In certain fusion protein embodiments, the invention provides a fusion protein comprising: mature protein sequence of SEQ ID No. 1 or 2, a detection or purification tag including a FLAG or His6 or Ig sequence; or sequence of another receptor protein.

Various kit embodiments include a kit comprising TLR14 protein or polypeptide, and: a compartment comprising the protein or polypeptide; and/or instructions for use or disposal of reagents in the kit.

Binding compound embodiments include those comprising an antigen binding site from an antibody, which specifically binds to TLR14 protein, wherein the protein is a primate protein; the binding compound is an Fv, Fab or Fab2 fragment; the binding compound is conjugated to another chemical moiety; or the antibody: is raised against a peptide sequence of a mature polypeptide to SEQ ID No. 1 or 2; is raised against a mature TLR14; is raised to a purified human TLR14; is immunoselected; is a polyclonal antibody; binds to a denatured TLR14; exhibits a Kd to antigen of at least 30 μM; is attached to a solid substrate, including a bead or plastic membrane; is in a sterile composition or is detectably labelled, including a radioactive or fluorescent label. A binding composition kit often comprises a binding compound and a compartment comprising said binding compound; and/or instructions for use or disposal of reagents in the kit. Often the kit is capable of making a qualitative or quantitative analysis.

Methods are provided for example of making an antibody comprising immunizing an immune system with an immunogenic amount of a primate TLR14, thereby causing said antibody to be produced, or producing an antigen/antibody complex comprising contacting such an antibody with a mammalian TLR14 protein or peptide thereby allowing the said complex to form.

Immunisation methods commonly practised in the art may be used and are well described in the literature.

Other compositions include a composition comprising: a sterile binding compound, or the binding compound and a carrier, wherein the carrier is an aqueous including water, saline and/or buffer, and/or formulated for oral, rectal, nasal, topical or parenteral administration.

Nucleic acid embodiments include an isolated or recombinant nucleic acid encoding a TLR14 or peptide or fusion protein, wherein the TLR is from a mammal; or the nucleic acid encodes an antigenic peptide sequence of SEQ ID No. 3 or 4; encodes a plurality of antigenic peptide sequences of SEQ ID No. 3 or 4; comprises at least 17 contiguous nucleotides from SEQ ID No. 3 or 4, exhibits at least 80% identity to natural cDNA encoding said segment; is an expression vector; further comprises an origin of replication; is from a natural source; comprises a detectable label such as a radioactive label, a fluorescent label, or an immunogenic label; comprises synthetic nucleotide sequence; is less than 6 kB, preferably less than 3 kB; is from a mammal, including a primate; comprises a natural full-length coding sequence; is a hybridisation probe for a gene encoding said TLR; or is PCR primer, PCR product, or mutagenesis primer. A cell, tissue or organ comprising such a recombinant nucleic acid is also provided. Preferably the cell is a prokaryotic cell; eukaryotic cell; bacterial cell; yeast cell; insect cell; mouse cell; mammalian cell; primate cell or human cell. Kits are provided comprising such nucleic acids and a compartment comprising said nucleic acid; a compartment further comprising a primate TLR14 protein or polypeptide; and/or instruction for use or disposal of reagents of the kit. Often the kit is capable of making a qualitative or quantitative analysis.

Also provided are methods for producing a ligand/receptor complex, comprising contacting a substantially pure TLR14 including a recombinant or synthetically produced protein with candidate TLR ligand, thereby allowing said complex to form.

A TLR ligand refers to a molecule that specifically binds to a TLR polypeptide, in this case aTLR14 polypeptide. In most cases the TLR ligand will also induce TLR signalling when contacted with the TLR under suitable conditions.

The invention also provides a method of modulating physiology or development of a cell or tissue culture cells comprising contacting the cell with an agonist or antagonist of a mammalian TLR14.

The present invention relates to methods of identifying and evaluating reagents that modulate the activity of TLR14 using at least one of the following as a marker: (i) NFkappaB activation (ii) NFkappaB protein or polynucleotide encoding the same (iii) IRF3 protein or polynucleotide encoding the same (iv) p38 protein or polynucleotide encoding the same (v) IKKs protein or polynucleotide encoding the same (vi) RANTES protein or polynucleotide encoding the same (vii) TLR4 protein or polynucleotide encoding the same or (viii) any pro-inflammatory or inhibitory cytokine.

The present invention also relates to the use of a reagent that alters the expression, amount, activity or phosphorylation, in a cell or tissue of (i) NFkappaB activation (ii) NFkappaB protein or polynucleotide encoding the same (iii) IRF3 protein or polynucleotide encoding the same (iv) p38 protein or polynucleotide encoding the same (v) IKKs protein or polynucleotide encoding the same (vi) RANTES protein or polynucleotide encoding the same (vii) TLR4 protein or polynucleotide encoding the same or (viii) any pro-inflammatory or inhibitory cytokine.

The present invention is based on the discovery of the novel TLR14 protein, and that the inhibition or activation of TLR14 can be detected by determining the amount, expression activity or phosphorylation of signal molecules which can lead to the activation of (i) NFkappaB activation (ii) NFkappaB protein or polynucleotide encoding the same (iii) IRF3 protein or polynucleotide encoding the same (iv) p38 protein or polynucleotide encoding the same (v) IKKs protein or polynucleotide encoding the same (vi) RANTES protein or polynucleotide encoding the same (vii) TLR4 protein or polynucleotide encoding the same or (viii) any pro-inflammatory or inhibitory cytokine.

One embodiment of the invention provides a method for monitoring the effect of TLR14 activation or inhibition by determining the difference in a level relative to a test sample of (i) NFkappaB activation (ii) NFkappaB protein or polynucleotide encoding the same (iii) IRF3 protein or polynucleotide encoding the same (iv) p38 protein or polynucleotide encoding the same (v) IKKs protein or polynucleotide encoding the same (vi) RANTES protein or polynucleotide encoding the same (vii) TLR4 protein or polynucleotide encoding the same or (viii) any pro-inflammatory or inhibitory cytokine.

“Level” used herein includes but not limited to, the amount of a protein, expression amount of mRNA, a gene activity, a protein activity, and the amount of phosphorylation.

Test samples may include but are not limited to peptide nucleic acids (PNAs), antibodies, polypeptides, carbohydrates, lipids, hormones and small molecules. Test compounds may also include variants of a reference immunostimulatory nuclei acid. These may be obtained from natural nucleic acid sources genomic nuclear or mitochondrial DNA or cDNA) or are synthetic (produced by oligonucleotide synthesis for example).

Thus in one aspect, the invention relates to methods for identifying and evaluating reagents that activate or inhibit TLR14 activity comprising, determining the difference in the amount, expression, activity or phosphorylation relative to a test sample of at least one of the following: (i) NFkappaB activation (ii) NFkappaB protein or polynucleotide encoding the same (iii) IRF3 protein or polynucleotide encoding the same (iv) p38 protein or polynucleotide encoding the same (v) IKKs protein or polynucleotide encoding the same (vi) RANTES protein or polynucleotide encoding the same (vii) TLR4 protein or polynucleotide encoding the same or (viii) any pro-inflammatory or inhibitory cytokine.

In another embodiment, such methods comprises determining the difference in the amount relative to a test sample of at least 2, at least 3, of each of (i) to (viii) as defined supra.

In one embodiment of the invention the difference in the amount relative to a test sample of mRNA is determined and can, for example, be determined by the use of nucleic acid microarrays.

In one embodiment of the invention the difference in the amount relative to a test sample of protein is determined.

Another aspect of the invention relates to a method for identifying or evaluating reagents that modulate the activity of TLR14, said method comprises: (i) NFkappaB activation (ii) NFkappaB protein or polynucleotide encoding the same (iii) IRF3 protein or polynucleotide encoding the same (iv) p38 protein or polynucleotide encoding the same (v) IKKs protein or polynucleotide encoding the same (vi) RANTES protein or polynucleotide encoding the same (vii) TLR4 protein or polynucleotide encoding the same or (viii) any pro-inflammatory or inhibitory cytokine. In another embodiment, such methods comprises determining the difference in the amount relative to a test sample of at least 2, at least 3, of each of (i) to (viii) as defined supra.

In a preferred embodiment of a method for identifying or evaluating reagents that modulate the activity of TLR14, said method comprises: : (i) NFkappaB activation (ii) NFkappaB protein or polynucleotide encoding the same (iii) IRF3 protein or polynucleotide encoding the same (iv) p38 protein or polynucleotide encoding the same (v) IKKs protein or polynucleotide encoding the same (vi) RANTES protein or polynucleotide encoding the same (vii) TLR4 protein or polynucleotide encoding the same or (viii) any pro-inflammatory or inhibitory cytokine. In another embodiment, such methods comprises determining the difference in the amount relative to a test sample of at least 2, at least 3, of each of (i) to (viii) as defined supra.

Sequence Homology

A particularly preferred nucleotide sequences of the invention is the human sequence set forth in SEQ ID NO:l or SEQ ID NO:2. The sequence of the amino acids encoded by the DNA of SEQ ID NO:3 is shown in SEQ ID NO:1. The sequence of the amino acids encoded by the DNA of SEQ ID NO:4 is shown in SEQ ID NO:2.

Due to the known degeneracy of the genetic code, wherein more than one codon can encode the same amino acid, a DNA sequence can vary from that shown in SEQ ID NO:3, and still encode a polypeptide having the amino acid sequence of SEQ ID NO:1. Such variant DNA sequences can result from silent mutations (e.g., occurring during PCR amplification), or can be the product of deliberate mutagenesis of a native sequence.

The invention thus provides isolated DNA sequences encoding polypeptides of the invention, selected from: (a) DNA comprising the nucleotide sequence of SEQ ID NO:1 (b) DNA encoding the polypeptide of SEQ ID NO:3 (c) DNA capable of hybridization to a DNA of (a) or (b) under conditions of moderate stringency and which encodes polypeptides of the invention; (d) DNA capable of hybridization to a DNA of (a) or (b) under conditions of high stringency and which encodes polypeptides of the invention, and (e) DNA which is degenerate as a result of the genetic code to a DNA defined in (a), (b), (c), or (d) and which encode polypeptides of the invention. Of course, polypeptides encoded by such DNA sequences are encompassed by the invention.

The invention thus provides equivalent isolated DNA sequences encoding biologically active human interferon alpha 14 polypeptides selected from: (a) DNA derived from the coding region of a native mammalian interferon alpha 14 allele c gene; (b) DNA of SEQ ID NO:3, (c) DNA capable of hybridization to a DNA of (a) or (b) under conditions of moderate stringency and which encodes biologically active interferon alpha 14 polypeptides; and (d) DNA that is degenerate as a result of the genetic code to a DNA defined in (a), (b) or (c), and which encodes biologically active interferon alpha 14 polypeptides.

As used herein, conditions of moderate stringency can be readily determined by those having ordinary skill in the art based on, for example, the length of the DNA. The basic conditions are set forth by Sambrook et al. Molecular Cloning: A Laboratory Manual, 2 ed. Vol. 1, pp. 1.101-104, Cold Spring Harbor Laboratory Press, (1989). Conditions of high stringency can also be readily determined by the skilled artisan based on, for example, the length of the DNA.

Also included as an embodiment of the invention is DNA encoding polypeptide fragments and polypeptides comprising inactivated N-glycosylation site(s), inactivated protease processing site(s), or conservative amino acid substitution(s).

In another embodiment, the nucleic acid molecules of the invention also comprise nucleotide sequences that are at least 80% identical to a native sequence. Also contemplated are embodiments in which a nucleic acid molecule comprises a sequence that is at least 90% identical, at least 95% identical, at least 98% identical, at least 99% identical, or at least 99.9% identical to a native sequence.

The percent identity may be determined by visual inspection and mathematical calculation. Alternatively, the percent identity of two nucleic acid sequences can be determined by comparing sequence information using the GAP computer program, version 6.0 described by Devereux et al. (Nucl. Acids Res. 12:387, 1984) and available from the University of Wisconsin Genetics Computer Group (UWGCG). The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps. Other programs used by one skilled in the art of sequence comparison may also be used.

The invention also provides isolated nucleic acids useful in the production of polypeptides. Such polypeptides may be prepared by any of a number of conventional techniques. A DNA sequence encoding an interferon alpha 14 polypeptide, or desired fragment thereof, may be subcloned into an expression vector for production of the polypeptide or fragment. The DNA sequence advantageously is fused to a sequence encoding a suitable leader or signal peptide. Alternatively, the desired fragment may be chemically synthesized using known techniques. DNA fragments also may be produced by restriction endonuclease digestion of a full length cloned DNA sequence, and isolated by electrophoresis on agarose gels. If necessary, oligonucleotides that reconstruct the 5′ or 3′ terminus to a desired point may be ligated to a DNA fragment generated by restriction enzyme digestion. Such oligonucleotides may additionally contain a restriction endonuclease cleavage site upstream of the desired coding sequence, and position an initiation codon (ATG) at the N-terminus of the coding sequence.

The well-known polymerase chain reaction (PCR) procedure also may be employed to isolate and amplify a DNA sequence encoding a desired protein fragment. Oligonucleotides that define the desired termini of the DNA fragment are employed as 5′ and 3′ primers. The oligonucleotides may additionally contain recognition sites for restriction endonucleases, to facilitate insertion of the amplified DNA fragment into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, innis et al., eds., Academic Press, Inc. (1990).

The invention encompasses polypeptides and fragments thereof in various forms, including those that are naturally occurring or produced through various techniques such as procedures involving recombinant DNA technology. For example, DNAs encoding interferon alpha 14 polypeptides can be derived from SEQ ID NO:3 by in vitro mutagenesis, which includes site-directed mutagenesis, random mutagenesis, and in vitro nucleic acid synthesis. Such forms include, but are not limited to, derivatives, variants, and oligomers, as well as fusion proteins or fragments thereof.

The polypeptides of the invention include full length proteins encoded by the nucleic acid sequence of SEQ ID NO:1. A particularly preferred polypeptide comprises the amino acid sequence of SEQ ID NO:3.

The polypeptides of the invention may be membrane bound or they may be secreted and thus soluble. Soluble polypeptides are capable of being secreted from the cells in which they are expressed. In general, soluble polypeptides may be identified (and distinguished from non-soluble membrane-bound counterparts) by separating intact cells which express the desired polypeptide from the culture medium, e.g., by centrifugation, and assaying the medium (supernatant) for the presence of the desired polypeptide. The presence of polypeptide in the medium indicates that the polypeptide was secreted from the cells and thus is a soluble form of the protein.

Also provided herein are polypeptide fragments of varying lengths. Naturally occurring variants as well as derived variants of the polypeptides and fragments are also provided herein.

The invention further relates to a pharmaceutical composition. The composition comprises: (a) a reagent that modulates the activity of a TLR14 polypeptide or polynucleotide and (b) a pharmaceutically acceptable carrier. The reagent may be a TLR14 agonist or antagonist. The composition may be used to treat the diseases such as an allergic disease, autoimmune disease, inflammatory disease, cardiovascular disease, Central Nervous System disease, neoplastic disease and infectious disease.

One skilled in the art will know that the choice of pharmaceutical carrier includes physiologically suitable compounds and the choice of compound depends on the route of administration and the intended administration regime.

Treatment/Therapy

The term ‘treatment’ is used herein to refer to any regimen that can benefit a human or non-human animal. The treatment may be in respect of an existing condition or may be prophylactic (preventative treatment). Treatment may include curative, alleviation or prophylactic effects.

More specifically, reference herein to “therapeutic” and “prophylactic” treatment is to be considered in its broadest context. The term “therapeutic” does not necessarily imply that a subject is treated until total recovery. Similarly, “prophylactic” does not necessarily mean that the subject will not eventually contract a disease condition.

Accordingly, therapeutic and prophylactic treatment includes amelioration of the symptoms of a particular condition or preventing or otherwise reducing the risk of developing a particular condition. The term “prophylactic” may be considered as reducing the severity or the onset of a particular condition. “Therapeutic” may also reduce the severity of an existing condition.

The present invention describes methods which involve unless otherwise indicated, commonly used techniques of cell biology, cell culture, molecular biology, transgenic biology, microbiology, recombinant DNA techniques and immunology, all of which art well described in the field.

The present invention further relates to an endogenous ligand(s) to TLR14 identified in and purified from cell and tissue extracts prepared from mammalian cells.

The present invention further relates to the modulation of TLR4 signalling, where TLR14 promotes or inhibits TLR4 signalling.

The peptides according to the present invention may be used in screening for molecules which affect or modulate activity or function of the peptides. The interaction of such molecules with the peptides may be useful in a therapeutic and prophylactic context.

It is well known that pharmaceutical research leading to the identification of a new drug may involve the screening of a very large number of candidate substances, both before and even after a lead compound has been found. Such means for screening for substances potentially useful in treating or preventing cancer. Substances identified as modulators of the polypeptide represent an advance in the therapy in these areas as they provide basis for design and investigation of therapeutics for in vivo use.

In various further aspects, the present invention relates to screening and assay methods and to substances identified thereby.

Thus, a further aspect of the present invention provides the use of a peptide (including a fragment or derivative thereof) of the invention in screening or searching for and/or obtaining or identifying a substance such as a peptide or chemical compound which interacts with or binds with the peptide of the invention and/or interferes with its biological function or activity or that of another substance. For instance, a method according to one aspect of the present invention includes providing a peptide of the invention and bringing it into contact with a substance, which contact may result in binding between the peptide and the substance. Binding may be determined by any number of techniques, both qualitative and quantitative which would be known to the person skilled in the art.

A substance identified as a modulator of peptide function may be a peptide or non-peptide in nature. Non-peptide “small molecules” are often preferred for many in-vivo pharmaceutical uses. Accordingly, a mimetic or mimic of the substance may be designed for pharmaceutical uses. The designing of mimetics to a known pharmaceutically active compound is a known approach to the development of pharmaceuticals based on a “lead” compound. This might be desirable where the active compound is difficult or expensive to synthesise or where it is unsuitable for a particular method of administration, e.g. peptides are not well suited as active agents for oral compositions as they tend to be quickly degraded by proteases in the alimentary canal. Mimetic design, synthesis and testing may be used to avoid randomly screening large number of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been determined, its structure is modelled according to its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can also be used in this modelling process.

In a variant of this approach, the three-dimensional structure of the ligand and its binding partner are modelled. This can be especially useful where the ligand and/or binding partner change conformation on binding, allowing the model to take account of the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in-vivo, while retaining the biological activity of the lead compound. The mimetic or mimetics found by this approach can then be screened to see whether they have the target property, or to what extent they exhibit it. Further optimisation or modification can then be carried out to arrive at one or more final mimetics for in-vivo or clinical testing.

A further aspect of the present invention therefore provides an assay for assessing binding activity between at least one peptide of the invention and a putative binding molecule which includes the steps of: bringing at least one peptide into contact with a putative binding molecule or other test substance, and determining interaction or binding between the at least one peptide and the binding molecule or test surface, wherein binding between the at least one peptide and the binding molecule is indicative of the utility of the at least one peptide.

A substance which interacts with the peptide of the present invention may be isolated and/or purified, manufactured and/or used to modulate its activity.

It is not necessary to use the entire peptide of the invention for assays of the invention which test for binding between two molecules. Fragments may be generated and used in any suitable way known to the person skilled in the art.

Further, the precise format of the assay of the invention may be varied by those skilled in the art using routine skill and knowledge.

### BRIEF DESCRIPTION OF THE DRAWINGS

The invention will be more clearly understood from the following description thereof given by way of example only with reference to the accompanying drawings in which:

FIG. 1A is a schematic representation of the chromosomal location of human TLR14. TLR14 is located on chromosome 7 at 7p15 as indicated by the line. It is 4.7 kb in length and is flanked by the genes CREB5 and CPVL. The direction of transcription is indicated by the arrows, TLR14 is transcribed in the anti-parallel direction. This information was obtained using the human genome map viewer tool available from the NCBI website at www.ncbi.nlm.nih.gov;

FIG. 1B shows the nucleotide sequences for human TLR14 (SEQ ID No. 1);

FIG. 1C shows the nucleotide sequences for murine TLR14 (SEQ ID No. 2);;

FIG. 1D shows the predicted protein sequence of human (SEQ ID No. 3); and murine (SEQ ID No. 4);TLR14. The putative ORF of the human TLR14 gene encodes an 811 amino acid protein while the murine protein is 809 amino acids in length. The predicted N-terminal signal sequence and transmembrane domains are underlined;

FIG. 1E shows the alignment of TLR4 and TLR14 ectodomains. Alignment of the putative TLR with human TLR4 reveals a high degree of sequence similarity between the two receptors. At least six leucine rich repeats can be identified and are highlighted by boxes;

FIG. 2A is an mRNA expression profile of human TLR14 expressed in several tissues. Expression profiles for the human and murine form of the novel TLR are available from the HUGE protein database. RT-PCR reactions were performed with primers targeting the 3′untranslated region of the mRNA encoding the protein. Expression was detected in all tissues tested with highest levels occurring in the kidney; brain and ovary;

FIG. 2B is a protein expression profile of TLR14 in human tissue samples. High expression levels were detected in the brain and lung.

FIG. 3 shows the alignment of the cytoplasmic region of TLR14 with other members of the TLR family. Alignment of the cytoplasmic region of TLR with other TLR family members reveals that the putative receptor shares regions of similarity that are characteristic of TLRs. Two regions in particular are homologous (see Box 1 and 2) and are considered the signature sequence of all TIR domain containing proteins. Box 2 of TLR14 is identical to that of TLR3;

FIG. 4 is a schematic representation of the putative promoter region of human TLR14. The putative promoter region of TLR14 was identified using Promoter Inspector and Mat inspector. All the transcription factors above have a matrix score* of greater than 0.8

*The matrix score measures how closely the sequences within the promoter correspond to the conserved nucleotides within the transcription factor matrix. A significant match is >0.8;

FIG. 5 shows the expression of TLR14 is induced by LPS in U373s and primary murine embryonic fibroblasts and also in mice treated with LPS. (A) U373s and MEFs were treated with 1 μg/ml LPS for the indicated times. mRNA was isolated and RT-PCR was carried out as described in the text. (B). Mice were injected with interperitoneally with LPS and left for 3 hours before being sacrificed. RT-PCR was carried out on control untreated and LPS treated mice.

FIG. 6 shows expression of TLR14 protein in cells following treatment with TLR ligands. (A) The human glioma cell line, A172, was treated for various times with Pam₃Cys (1 μg/ml) and probed for expression of TLR14. (B) Human HEK-293 cells stably transfected with TLR4 were treated with LPS (1 μg/ml) for various times and probed for expression of TLR14. (C) Protein extracts were prepared from the brains of control untreated mice and mice that had been injected with LPS. The extracts were probed for expression of TLR14.

FIG. 7 are graphs showing TLR14 activity induces of NF-κB- and ISRE-reporter gene expression in HEK293 and U373 astrocytoma cells. TLR14 activity drives NF-κB- and ISRE-luciferase activity in HEK293 and U373 astrocytoma cells. HEK293 cells were transfected with the NF-κB reporter construct along with 1, 5 and 10 ng of TLR14 (A). HEK293s (B) and U373s (C) were transfected with an ISRE reporter construct and increasing doses (1, 10 and 100 ng) of TLR14. After 24 h the cells were harvested and relative luciferase activity was determined; and

FIG. 8 is a graph showing TLR14 drives Rantes production in U373 astrocytoma cells. RANTES production was measured by Enzyme-Linked lmmunoabsorbant Assay in U373 cells that had been transfected for 24 h with increasing doses of TLR14. Data are expressed as fold induction over cells transfected with empty vector.

FIG. 9 shows interactions between TLR14 and the TIR-domain contain proteins TLR2, TLR4 and MyD88.(A) TLR14 was co-tranfected into HEK-293 cells together with Flag-tagged TLR4, TLR2 or mutant forms of the receptors. The complexes were immunprecipitated with anti-flag beads and probed with an anti-TLR14 antibody. (B) TLR14 was co-tranfected into HEK-293 cells together with Myc-tagged MyD88. The complex was immunprecipitated with an anti-myc antibody coupled to protein-A sepharose beads and probed with an anti-TLR14 antibody.

FIG. 10. shows an interaction between TLR2 and endogenous TLR14. Flag-tagged TLR2 was immunoprecipitated from HEK-293 cells and western blots were probed with an anti-TLR14 antibody to detect presence of the endogenous protein in complex with TLR2.

FIG. 11 shows that TLR14 is present in the cytosol and is also found at high levels in serum. (A) Cells were stimulated with LPS before being separated into cytosolic and membrane fraction. The fractions were probed for the presence of TLR14. (B) Cell culture medium containing 10% fetal calf serum was subject to western blotting and probed for the presence of TLR14.

FIG. 12 shows the secretion of TLR14 into U373 culture medium following stimulation of the cells with LPS (1 μg/ml) for the indicated time points. The secreted protein appears to be the full length form of TLR14 with maximum secretion occurring at 6 hours.

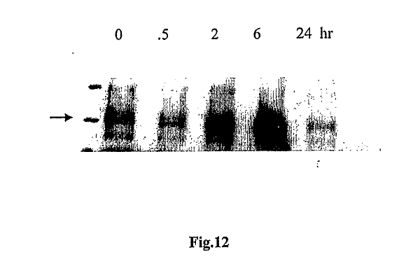

### DETAILED DESCRIPTION

We have identified a novel gene that shows remarkable homology with members of the Toll-like receptor/Interleukin-1 receptor (TLR) family. In cell-based assays, this novel receptor activates the transcription factors NF-κB and IRF3 and drives the production of the anti-viral cytokine, RANTES. The protein interacts with the TLR2, TLR4 and the universal TLR adapter, MyD88. We have named the receptor TLR14.

Expression of this putative receptor is enhanced by microbial products, for example LPS, suggesting that it may function as an immuno-modulator. In support of this, the transcription factors NF-κB and IRF3 were activated when cells were transfected with a vector expressing TLR14. As both NF-κB and IRF3 are central in the elimination of bacterial and viral pathogens, inhibiting or activating TLR14 is a promising new approach for the treatment of inflammatory diseases. In addition, we have found high levels of TLR14 in serum. A soluble form of TLR2 comprising mainly of the ectodomain of this receptor is also found at high levels in serum and in breast milk. This form of TLR2 is protective in that it dampens over active immune responses to TLR2 ligands. The full length TLR14 polypeptide or the ectodomain itself may have similar biological properties and could therefore be considered a potential biotherapeutic.

A microarray approach was used to identify genes that are regulated by LPS and components of the TLR4 signalling pathway. As mentioned above, the adapter molecule Mal is required to transmit signals from TLR2 and TLR4 following receptor stimulation. We used a gene-targeting vector to disrupt the gene encoding Mal in embryonic stem cells. These cells were then treated with LPS and differences in gene expression between knockout and wild-type cells were measured. In this way the gene that shows remarkable homology with members of the Toll-like receptor/Interleukin-1 receptor (TLR) family was identified.

The examples presented are illustrative only and various changes and modifications within the scope of the present invention will be apparent to those skilled in the art.

Materials & Methods

Cell Culture.

HEK 293 and U373 cells were cultured in Dulbecco's Modified Eagles Medium (DMEM) with 10% fetal bovine serum (FBS), supplemented with 100 units/ml penicillin, 100 mg/ml streptomycin, and 2 mM L-glutamine.

Expression Plasmids.

The chimeric TLR receptor CD4-TLR4, was a gift from R. Medzhitov (Yale University, New Haven, Conn.). The vector containing the TLR14 cDNA (KIAA0644) was supplied by the Kazusa DNA Research Institute and used as target for subsequent PCR cloning. The primers used included restriction sites for HindIII and EcoRV and were as follows: 5′-GCAAGCTTATGGAGGCTGCCCGCGCCTTG (sense) (SEQ ID No. 5); and 5′-GCGATATCGGCCTAAGCGTAGTCTGGGACGTCGTATGGGTAGTCGGCAAATC GC (antisense) (SEQ ID No. 6);. The antisense primer includes a sequence encoding a 9 amino acid hemagglutinin epitope tag in order to detect expression of the translated protein product in transfected cells. The resulting EcoR1-HindIII fragment was ligated into the multiple cloning site of the mammalian expression vector pCDNA 3.1 (Invitrogen).

Generation of Mal Deficient Embryonic Stem Cells and Microarray Analysis.

Embryonic stem cells lacking the gene encoding Mal were generated by homologous recombination. Briefly, murine embryonic stem cells were electroporated with a targeting vector, in which a 700 by exon encoding most of the coding sequence of the Mal gene was replaced with a neomycin resistance cassette. Targeted cells were identified by southern blotting before being subjected to a second round of targeting in order to generate clones homozygous for the Mal deletion. Mutant and wild-type cells were stimulated with LPS for various times and RNA was extracted for microarray analysis.

Promoter Analysis.

The complete nucleotide sequence of the human Riken clone KIAA0644 and flanking regions was obtained from the National Center for Biotechnology Information (NCBI) website at www.ncbi.nlm.nih.gov. Identification of transcribed nucleotide sequences and repeat sequences in the genomic sequence was performed using the NIX application (http://menu.hgmp.mrc.ac.uk) and the program Repeat-masker (http://searchlauncher.bcm.tmc.edu) (20). Transcription factor binding site predictions were performed using MatInspector Release Professional (www.genomatix.de/cgi-bin/matinspector/matinspector.pI) (21).

mRNA Isolation from Cultured Cells.

mRNA was extracted from cells following treatment for various times with LPS (1 μg/ml). Briefly, treated cells were pelleted and lysed in l ml of TRI reagent (Sigma). Chloroform (0.2 ml) was added to the sample and the mixture was centrifuged at 12,000 g for 15 minutes. The RNA containing aqueous phase was removed and the total RNA was precipitated from the mixture with the addition of an equal volume of isopropanol. Following centrifugation at 12,000 g for 10 minutes, the RNA containing pellet was washed with 500 μl of 75% ethanol. Any traces of ethanol were then removed and the pellet was left to dry at room temperature for 10 minutes. The pellet was resuspended in 30 μl of RNAse free water and stored at −80° C.

Reverse Transcriptase Polymerase Chain Reaction (RT-PCR).

RT-PCR was carried out using the Promega ImpromptII RT-PCR kit. The reverse transcription reaction was carried out in two steps, a PCR reaction was then carried out on the synthesised cDNA.

Step 1: 1 μl of Random Primers were added to 4 μl of RNA in a thin walled 500 μl PCR micro centrifuge tube. The tube was placed in a thermal cycler at 70° C. for 5min and 4° C. for 5 min.

Step 2: A second set of components were added; 1 μl deoxynucleotide mix (dNTPs mix) (500 μM each dNTP), 5.5 μl of PCR reagent water, 4.0 μl of 10× buffer, 3.0 μl of magnesium chloride, 1 μl RNase inhibitor (1 units/μl), 1 μl of RT (1 units/μl). This brought the total volume of the PCR tube to 20 μl. The tube was placed in a thermal cycler for the following times and temperatures, 25° C. for 5 min, 42° C. for 60 min, 70° C. for 15 min.

The following was added to a thin walled 500 μl PCR microcentrifuge tube on ice: 5 μl of 10× buffer, 1 μl dNTPs (200 μM each dNTP), 1 μl PCR primers (0.4 μof each), 2-5 μl Template DNA (cDNA), 1 μl Taq DNA polymerase mix (0.05 units/μl) and a sufficient volume of PCR reagent water to make a total volume in the PCR tube of 50 μl. The amplification temperatures were as follows, denaturation/RT inactivation (step 1) 94° C. for 2 min, denaturation (step 2) 94° C. for 15 sec, annealing (step 3) 55° C. for 30 sec, extension 68° C. for 1 min (step 2, 3 and 4 were repeated 35 times), final extension (step 5) 68° C. for 5 min. The PCR products were then electrophoresed on a 1% agarose gel and visualised on a UV transluminator.

Detection of Protein Expression.

A peptide antibody directed at the C-terminus of the putative protein was synthesised by Eurogentec, Liege Science Park, Belgium. The peptide used for immunization is composed of the following amino acids—CGSLRREDRLLQRFAD (SEQ ID No. 7);. Cell lines were treated for various times with TLR ligands as indicated. Stimulations were stopped with the addition of cold PBS and cells were lysed in SDS-PAGE sample buffer. For western blotting, the TLR14 antibody was diluted 1:1000 in tris buffered saline containing 0.5% tween 20.

Luciferase Reporter Gene Assays.

HEK 293 cells or U373 cells were seeded into 96-well plates (2×10⁴ cells per well) and transfected the next day with expression vector and reporter plasmids. Genejuice™ (Novagen) was used for transient transfections, according to the manufacturer's instructions. For experiments involving NF-κB or IRF3, 80 ng of the NF-κB- or ISRE-luciferase reporter gene (Stratagene) were transfected into cells along with 40 ng of the Renilla luciferase internal control plasmid (Promega). After 24 h cells were harvested in passive lysis buffer (Promega) and reporter gene activity was measured in a luminometer. In cases where cells were stimulated, LPS (Sigma) was added to the cells at a final concentration of 1 μg/ml 6 h prior to harvesting. Data are expressed as mean fold induction±s.d. relative to control levels, for a representative experiment from a minimum of three separate experiments, each performed in triplicate.

Enzyme-Linked Immunoabsorbant Assay.

U373 cells were transfected with increasing doses (1, 10 and 100 ng) of the TLR14 expression plasmid. The cells were incubated at 37° C. for 24 h. A 96 well microtitre plate was coated with the capture antibody (mouse anti-human RANTES) at a final concentration of 40 ng/ml. After 24 hours the plates were washed with PBS containing 0.05% Tween 20. The plates were then blocked for 1 h at room temperature in PBS containing 1% BSA and 5% sucrose. Cell supernantant (100 μl) was added to each well and the plates were incubated for 2 h at room temperature. Detection antibody (biotinylated goat anti-human RANTES) was then added to the wells at a final concentration of 10 ng/ml. The plates were again incubated for 2 h at room temperature. After washing, 100 μl of streptavidin-HRP was added to each well, the plates were covered and incubated for 20 minutes at room temperature. Finally, 100 μl of substrate solution (R&D Systems, Catalog #DY999) was added to the wells followed by 50 μl of stop solution (2N H₂SO₄). The optical density of each well was measured in a microplate reader set to 450 nm.

Co-Immunoprecipitation Assays.

HEK293 cells were seeded on 10 cm plates at 1×10⁵ cells/ml. The following day, cells were transfected with 3 μg of flag-tagged TLR2, TLR4 or Myc-tagged MyD88. After 24 hrs the cells were lysed in Hepes buffer containing 1% NP40. The cell lysates were then incubated with M2 anti-flag agarose beads (Sigma). After three hours the beads were washed x3 with Hepes buffer and resuspended with 20 μl of SDS-PAGE sample buffer. The protein samples were run on 10% SDS-PAGE gels and transferred to nitrocellulose for western blotting. The resulting blots were probed with anti-TLR14 and anti-flag antibodies.

Localisation Studies.

Cells were seeded in 10 cm dishes at 1×105 cells/ml 24 hours prior to stimulation with LPS. Membrane and cytosolic fractions were prepared by ultracentifugation and subjected to SDS-PAGE and western blotting in order to determine the localisation of TLR14. Medium (DMEM) containing 10% FCS was blotted for the presence of TLR14 following SDS-PAGE.

Characterization of the Gene Encoding TLR14.

Preliminary microarray analysis identified six genes that exhibit lower expression levels in Mal knockout cells. Five of the genes identified have been characterised to some extent while the remaining gene is novel and characterised herein. The sequence of this gene is available on the HUGE (Human Unidentified Gene-Encoded Large Proteins) protein database as part of the Human cDNA project at the Kazusa DNA Research Institute (www.Kazusa.or.jp). We have named this novel gene TLR14 for reasons outlined below.

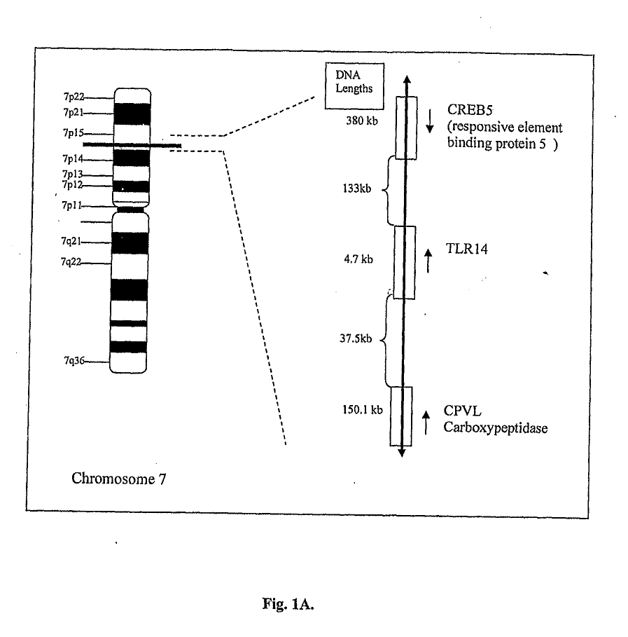

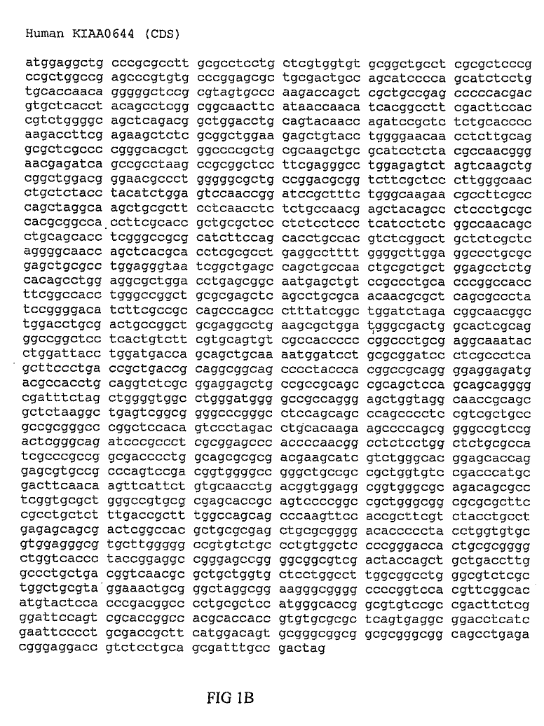

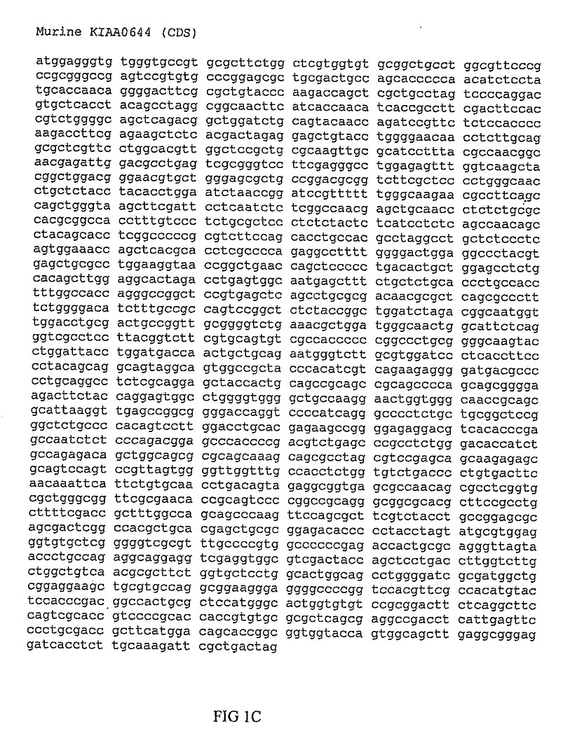

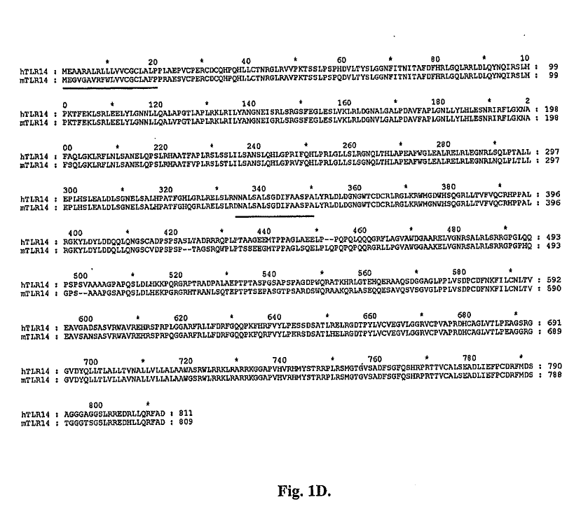

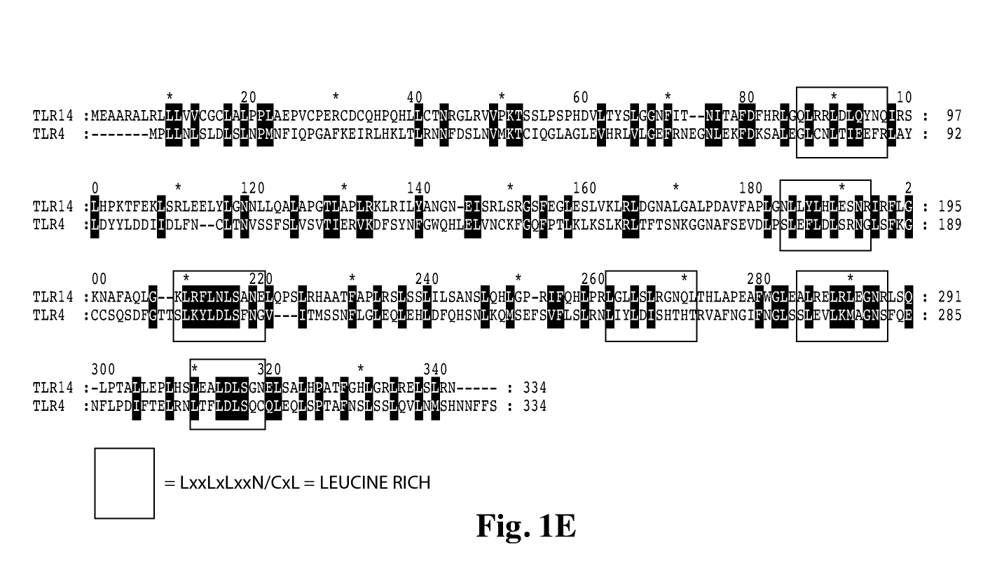

We have mapped the gene to human chromosome 7 using the Map Viewer tool available from NCBI (FIG. 1A). The gene is 4.7 kb in length and is flanked by CREB5 and CPVL carboxypeptidase. The nucleotide sequences for human and murine TLR14 are shown in FIGS. 1B and 1C, respectively. The predicted protein is 811 amino acids in length (FIG. 1D) and contains an N-terminal signal sequence, a feature common to all membrane localised proteins. The N-terminus of the putative protein also contains at least 6 leucine rich repeats and is highly homologous to the extracellular region of several TLRs (TLR4 is given as an example in FIG. 1E).

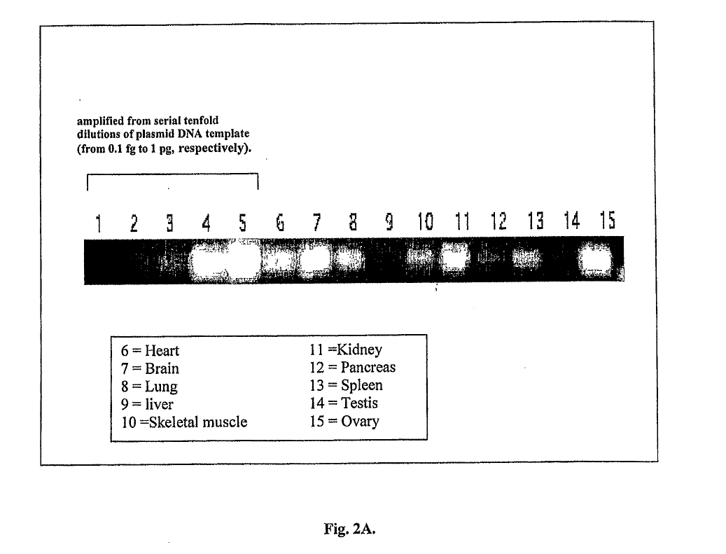

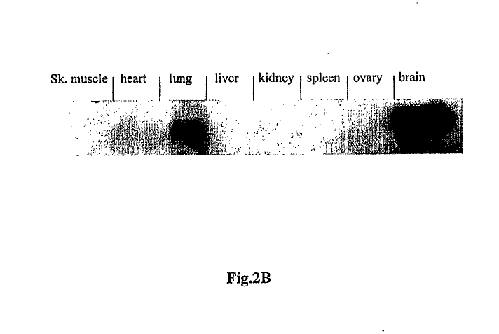

Expression profiles reveal a high abundance of the gene product in brain, kidney and ovary as shown in FIG. 2A (information obtained from Kazusa DNA Research Institute). We have generated a polyclonal antibody to the C-terminus of TLR14. The peptide used for immunization comprises the amino acids CGSLRREDDRLLQRFAD (SEQ ID No. 7);. The antibody detected a protein at approximately 81 kDa in human brain and lung tissue (FIG. 2B).

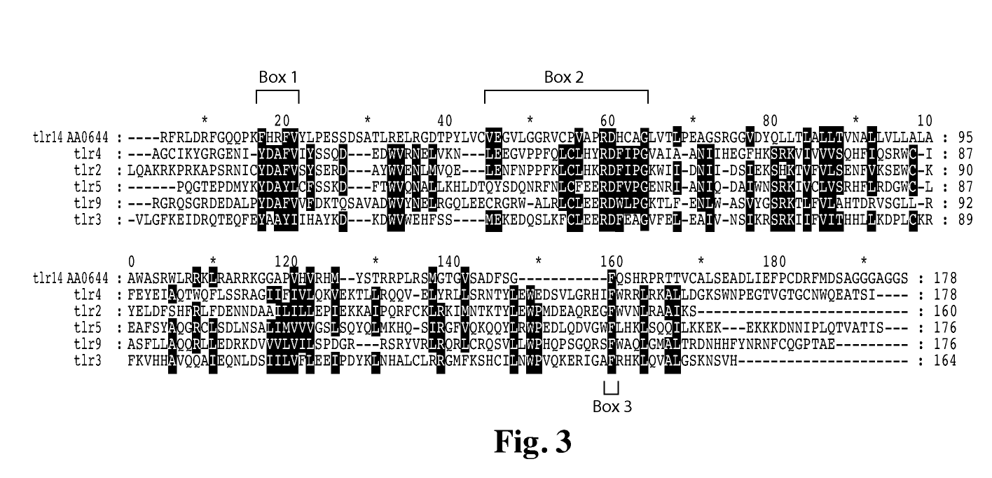

As described above, members of the TLR family all contain a cytosolic TIR domain. This domain spans about 200 amino acids, with varying degrees of sequence similarity among family members. Three particular boxes can be identified which are highly conserved among family members. Box1 is considered the signature sequence of the family whereas boxes 2 and 3 contain amino acids critical for signalling. The crystal structure of the TIR domains of TLR1 and TLR2 has revealed a core structural element centered around box 2 (22). This region, termed the BB loop, forms an exposed surface patch and contains a critical proline or arginine residue. These amino acids are located at the tip of the loop and are thought to form a point of contact with downstream signalling components. Close inspection of TLR14 reveals that it also contains a highly conserved box 2 and an identifiable box 1 and 3 (FIG. 3) suggesting that this novel protein belongs to the TLR superfamily.

Expression of TLR14 is Induced Following Treatment of Cells with TLR2 and TLR4 Ligands.

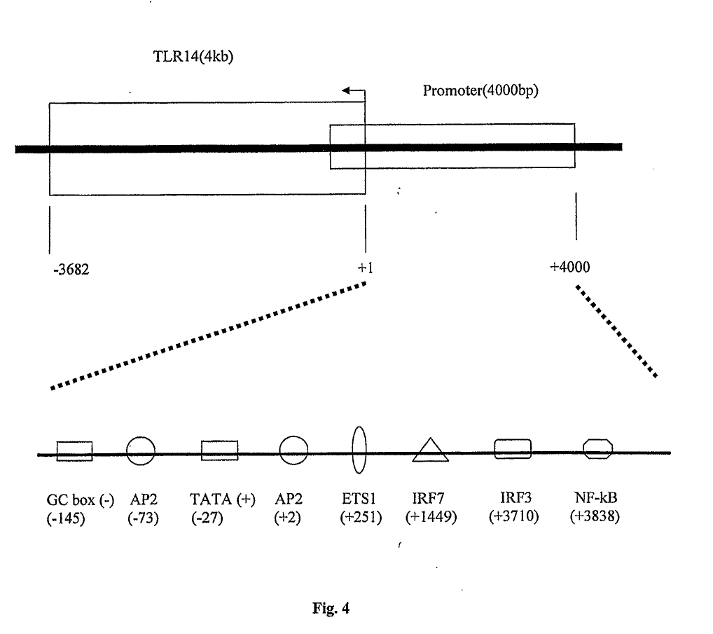

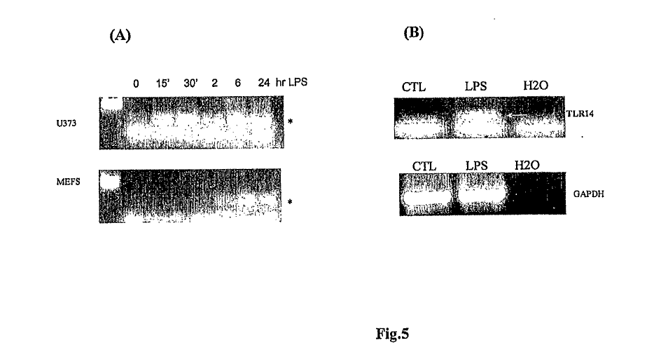

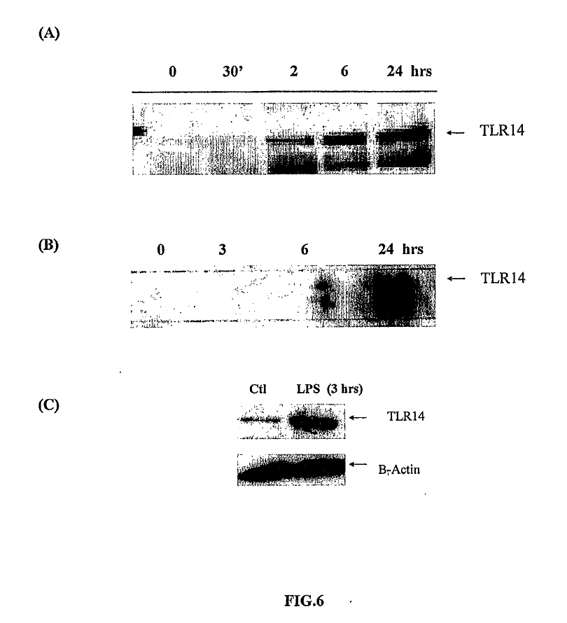

As described above, TLR14 expression was abolished in cells lacking Mal following exposure to LPS. This indicates that the gene in question is regulated by LPS and possibly other TLR ligands. In order to address this issue further, we identified the promoter region of TLR14 and possible transcription factor binding sites using the NIX application (http://menu.hgmp.mrc.ac.uk) and MatInspector Release Professional (www.genomatix.de/cgi-bin/matinspector/matinspector.pI). It is likely that the functional TLR14 promoter is contained within the 4 kb region proximal to exon 1. Further analysis of this region revealed putative binding sites for several transcription factors, such as NF-κB, IRF7 and Ets-1 (FIG. 4). The induction of TLR14 mRNA expression was analysed by RT-PCR following treatment of cells with inflammatory stimuli. As shown in FIG. 5A, TLR14 mRNA expression is induced in brain astrocytoma cells (U373s) and primary murine embryonic fibroblasts (MEFs) with time following exposure to LPS. A striking increase was also detected in the levels of TLR14 mRNA prepared from the brains of mice treated with LPS (FIG. 5B). Induction of expression was also detected at the protein level in the humal glioma cell line, A172, following treatment with the TLR2 ligand Pam₃Cys, as shown in FIG. 6A. A similar effect was seen in HEK-293 cells stably transfected with TLR4 following treatment with LPS (FIG. 6B). In addition, an increase in TLR14 protein expression was seen in the brains of mice injected with LPS as shown in FIG. 6C.

TLR14 Activates the Transcription Factors NF-κB and IRF3.

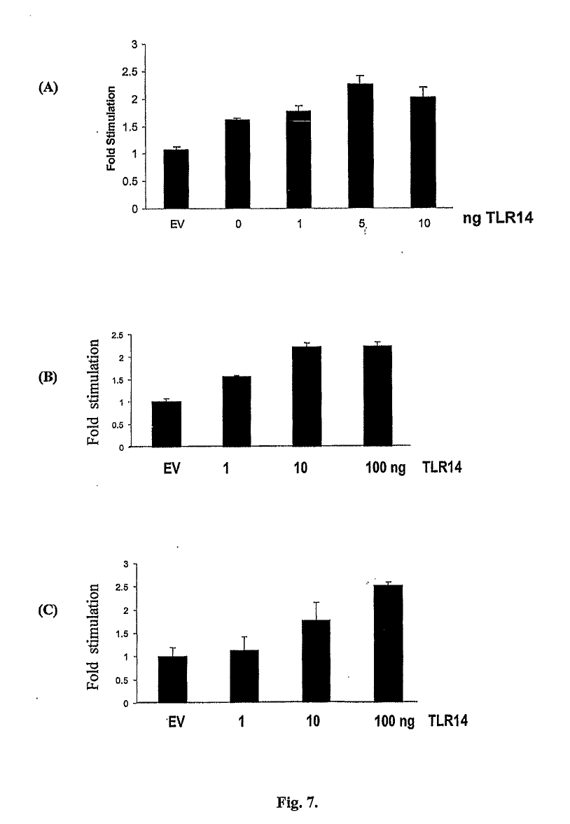

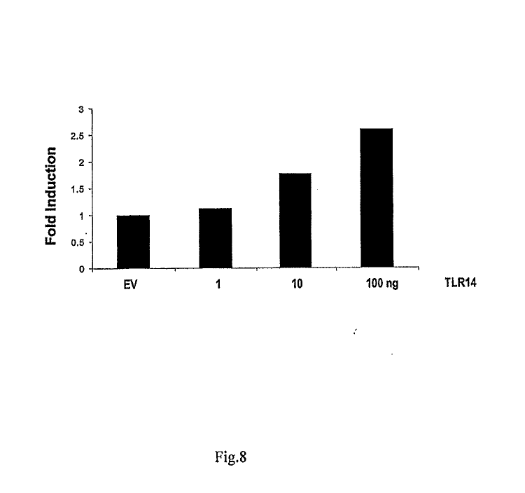

As described above, NF-κB is activated by most members of the TLR superfamily while IRF3 activation is restricted to TLR3 and TLR4. In order to address whether TLR14 can also activate these factors and therefore modulate immune responses, we cloned the cDNA encoding the protein into the mammalian expression vector pcDNA 3.1 and performed functional assays using luciferase reporter constructs containing elements of DNA to which NF-κB and IRF3 bind. The protein contains a tag encoding hemaglutinin (HA) and expression was detected in various cell lines using an anti-HA antibody (data not shown). When the TLR14 expression plasmid was transfected into cells along with the κB and ISRE reporter constructs, luciferase activity was enhanced (FIG. 7) suggesting that TLR14, like TLR4, activates both NF-κB and IRF3. Preliminary ELISAs have also shown an increase in RANTES production (an IRF3 inducible cytokine) in cells transfected with TLR14 (FIG. 8).

TLR14 Interacts with Other Members of the TLR Family.

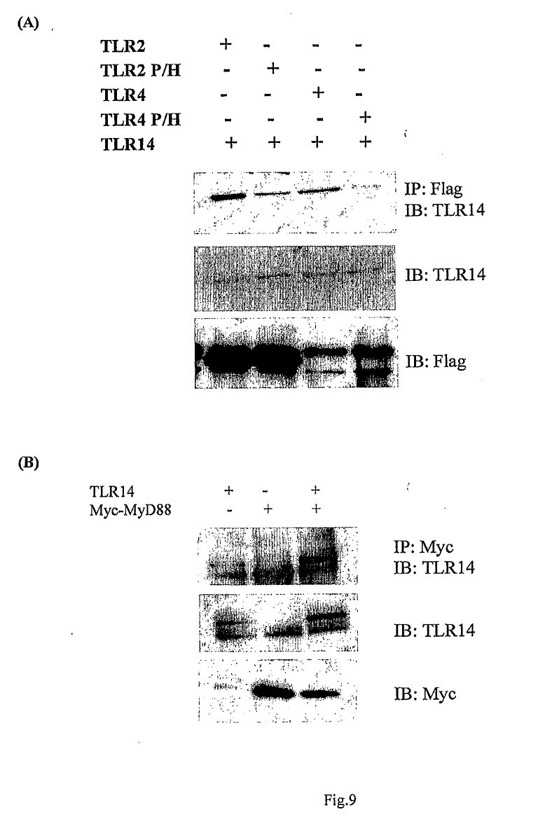

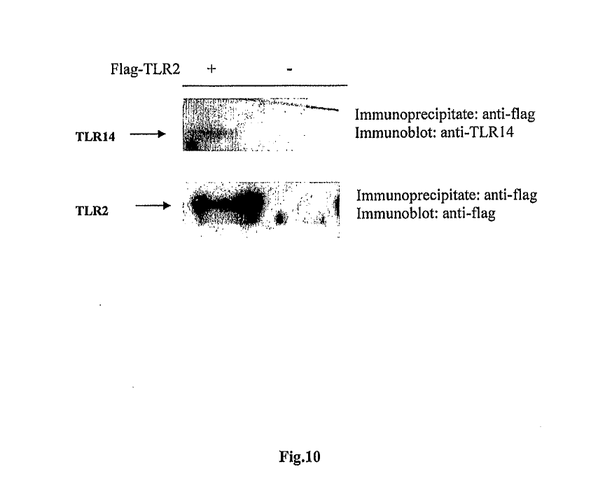

A common feature of TIR domain containing proteins is their ability to homo- or heterodimerize with other TIR domain containing proteins. We performed co-immunoprecipitation experiments with TLR14 and the TIR domain containing receptors TLR2 and TLR4 in order to determine if TLR14 could interact with either or both receptors. We found that TLR14 interacts strongly with overexpressed TLR2 and TLR4 as shown in FIG. 9A. Mutation of the conserved proline residue to a histidine in the TIR domain of TLRs is known to abolish TIR-TIR interactions (22). Accordingly, the interaction between TLR14 and either TLR2 or TLR4 was significant reduced with mutant (P/H) forms of the receptors were co-expressed with TLR14. TLR14 was also found to interact with the universal TIR-domain containing adapter MyD88 as shown in FIG. 9B. This supports the notion that TLR14 is a TIR domain containing protein. Finally, we were able to detect an interaction between TLR2 and endogenous TLR14 as shown in FIG. 10. In order to test this, we transfected HEK293 cells with flag-tagged TLR2. Cells were then lysed and incubated with anti-flag beads in order to immunprecipitate TLR2 and any interacting proteins. Following western blotting, we were able to detect a band corresponding to TLR14 using the anti-TLR14 antibody.

TLR14 is Found at high Levels in Serum and may be Produced as a Soluble Protein.

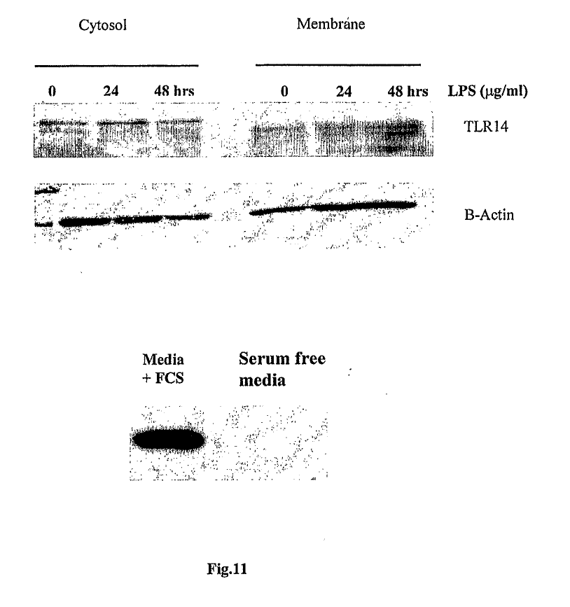

We prepared cellular fractions in order to determine whether TLR14 is localised to the plasma membrane. Surprisingly, TLR14 was found in the cytosolic fraction of cells (FIG. 11A). In addition, high levels of the protein were found in fetal calf serum (FIG. 11B) suggesting that the protein may be a soluble secreted protein. Mass spectroscopic analysis revealed that the band present in FCS was the bovine homolog of human TLR14 (data not shown). Preliminary experiments have also shown that the protein is secreted from U373 cells following stimulation with LPS. The protein does not appear to be cleaved as the molecular weight corresponds to that of the full length protein. Maximum secretion occurs at 6 hours.

The invention is not limited to the embodiments hereinbefore described which may be varied in detail.

### REFERENCES

1. Poltorak, A. et al. Science 282, 2085-2088 (1998).

2. Qureshi, S. T. et al. J. Exp. Med. 189, 615-625 (1999).

3. Hayashi, F. et al. Nature 410, 1099-1103 (2001).

4. Hemmi, H. et al. Nature 408, 740-745 (2000).

5. Zhang, D. et al. Science 303, 1522-1526 (2004).

6. Tabeta, K. et al. PNAS 101, 3516-3521 (2004)

7. Hemmi, H. et al. Nature Immunol. 3, 196-200 (2002).

8. Adachi, O. et al. Immunity 9, 143-150 (1998).

9. Takeuchi, O. et al, J. Immunol. 164, 554-557 (2000).

10. Yamamoto, M. et al. J. Immunol. 169, 6668-72 (2002).

11. Kaisho, T. et al. J. Immunol. 166, 5688-5694 (2001).

12. Servant, M. J. et al. J. Biochem. Pharmacol. 64, 985-992 (2002).

13. Fizgerald, K. A. et al. Nature 413, 78-83 (2001).

14. Homg, T. et al. Nature Immunol. 2, 835-841 (2001).

15. Yamamoto, M. et al. Nature 420, 324-329 (2002).

16. Horng, T. et al. Nature 420, 329-33 (2002).

17. Axtelle, T. & Pribble, J. J. Endotoxin Res. 7, 310-314 (2001).

18. Lynn, M. et al. J Infect Dis. 187, 631-639 (2003).

19. Berman, B. Int. J. Dermatol. 29, 7-11 (2002).

20. Smith, R. et al. Genome Res. 6, 454-462 (1996).

21. Quandt, K. et al. Nucleic Acids Res. 23, 4878-4884 (1995).

22. Xu, Y. et al. Nature 408, 111-5 (2000).

1-69. (canceled)

70. A method of treating an immune or inflammatory disorder, comprising: administering a therapeutically effective amount of a polypeptide comprising the amino acid sequence of SEQ ID No. 1 or 2, or a fragment or variant thereof, to a subject in need thereof.

71. The method of claim 70 wherein the fragment is chosen from one or more of: an antigenic fragment of SEQ ID NO: 1 or 2, the TLR14 ectodomain shown in FIG. 1E, or the cytoplasmic region of TLR14 shown in FIG. 3.

72. The method of claim 70 wherein the variant comprises an amino acid sequence that is at least 70% identical to the amino acid sequence of SEQ ID No. 1 or 2.

73. The method of claim 70 wherein the variant comprises a modification chosen from one or more of: a deletion modification, an insertion modification, or a post translational modification.

74. The method of claim 70 wherein the polypeptide or fragment or variant thereof is a soluble or a fusion protein.

75. The method of claim 74 wherein the polypeptide or fragment or variant thereof is capable of inhibiting the activity of TLR14.

76. An adjuvant or vaccine formulation wherein the adjuvant or vaccine formulation comprises a polypeptide comprising the amino acid sequence of SEQ ID No. 1 or 2, or a fragment or variant thereof.

77. A soluble or a fusion a polypeptide comprising the amino acid sequence of SEQ ID No. 1 or 2, or a fragment or variant thereof.

78. The soluble or fusion polypeptide of claim 77 wherein the fragment is chosen from one or more of: an antigenic fragment of SEQ ID No: 1 or 2, the TLR14 ectodomain shown in FIG. 1E or the cytoplasmic region of TLR14 shown in FIG. 3.

79. The soluble or fusion polypeptide of claim 77 wherein the variant comprises an amino acid sequence that is at least 70% identical to the amino acid sequence of SEQ ID No. 1 or 2.

80. The soluble or fusion polypeptide of claim 77 wherein the fragment or variant thereof is capable of inhibiting the activity of TLR14.

81. A method for identifying a compound which modulates Toll-like receptor activity comprising the steps of :
contacting a polypeptide comprising an amino acid sequence of SEQ ID No. 1 or 2 or variant or fragment thereof with a test sample comprising one or more compounds;
monitoring for markers of Toll-like receptor activity; and
identifying the one or more compounds which modulate Toll-like receptor activity.

82. The method of claim 81 wherein the fragment is chosen from one or more of: an antigenic fragment of SEQ ID NO: 1 or 2, the TLR14 ectodomain shown in FIG. 1E, or the cytoplasmic region of TLR14 shown in FIG. 3.

83. The method of claim 81 wherein the variant comprises an amino acid sequence that is at least 70% identical to the amino acid sequence of SEQ ID No. 1 or 2.

84. The method of claim 81 wherein the markers of Toll-like receptor activity comprise any one, two, three or more of:
(i) NFkappaB activation
(ii) NFkappaB protein or polynucleotide encoding the same
(iii) IRF3 protein or polynucleotide encoding the same
(iv) p38 protein or polynucleotide encoding the same
(v) IKKs protein or polynucleotide encoding the same
(vi) RANTES protein or polynucleotide encoding the same
(vii) TLR4 protein or polynucleotide encoding the same or
(viii) any pro-inflammatory or inhibitory cytokine.

85. The method of claim 81 wherein a compound which activates or inhibits TLR activity is identified by determining the amount, expression, activity or phosphorylation relative to the test sample of a least one or more of:
(i) NFkappaB activation
(ii) NFkappaB protein or polynucleotide encoding the same
(iii) IRF3 protein or polynucleotide encoding the same
(iv) p38 protein or polynucleotide encoding the same
(v) IKKs protein or polynucleotide encoding the same
(vi) RANTES protein or polynucleotide encoding the same
(vii) TLR4 protein or polynucleotide encoding the same or (viii) any pro-inflammatory or inhibitory cytokine.

86. A method of screening for a compound capable of inhibiting or promoting NF-κB activation comprising the steps of:
providing a host cell comprising a nucleic acid encoding a protein comprising the amino acid sequence of SEQ ID No. 1 or 2, or a variant or fragment thereof and a component that provides a detectable signal associated with activation of NF-κB;
culturing the host cell under conditions providing the expression of the nucleic acid in the host cell;
contacting the host cell with one or more compounds for screening;
measuring the detectable signal; and
isolating or identifying the one or more compounds as an activator compound or an inhibitor compound.

87. A method of preparing a pharmaceutical composition comprising the steps as claimed in claim 86 including the step of:
optimising the isolated or identified compound as a pharmaceutical compound.

88. The method of claim 86 wherein the fragment is chosen from one or more of:
an antigenic fragment of SEQ ID NO: 1 or 2, the TLR14 ectodomain shown in FIG. 1E, or the cytoplasmic region of TLR14 shown in FIG. 3.

89. The method of claim 86 wherein the variant comprises an amino acid sequence that is at least 70% identical to the amino acid sequence of SEQ ID No. 1 or 2.

90. The method of claim 86 wherein the protein is a recombinant protein comprising an amino acid sequence of SEQ ID No. 1 or 2.

91. An isolated nucleic acid comprising a nucleotide sequence of SEQ ID No: 3 or SEQ ID No: 4, or a variant or fragment thereof, or a nucleotide sequence complementary thereto.

92. A recombinant vector comprising the nucleic acid according to claim 91.

93. A host cell comprising the nucleic acid according to claim 91.

94. A method of producing a polypeptide comprising culturing the host cell of claim 93 under conditions where the polypeptide is expressed.

95. An antibody or fragment thereof that specifically binds to an epitope of a polypeptide comprising the amino acid sequence of SEQ ID No. 1 or 2, or a fragment or variant thereof.

96. A method of treating an immune or inflammatory disorder, comprising: administering a therapeutically effective amount of an antibody or fragment thereof that specifically binds to an epitope of a polypeptide comprising the amino acid sequence of SEQ ID No. 1 or 2, or a fragment or variant thereof, to a subject in need thereof.
